(19) United States
(12) Patent Application Publication
(10) Pub. No.: US 2017/0261485 A1
(43) Pub. Date: Sep. 14, 2017

(54) HYDROGEN SENSOR HAVING A PROTECTION LAYER

(71) Applicant: ABB Schweiz AG, Baden (CH)

(72) Inventors: Barbara Panella, Wettingen (CH); Jacobus Lodevicus Martinus van Mechelen, Regensdorf (CH); Ruud Johannes Westerwaal, Barsingerhorn (NL); Bernard Dam, Den Haag (NL)

(21) Appl. No.: 15/437,186

(22) Filed: Feb. 20, 2017

Related U.S. Application Data
(63) Continuation of application No. PCT/EP2015/068826, filed on Aug. 17, 2015.

(30) Foreign Application Priority Data
Aug. 19, 2014 (EP) 14181426.9

(51) Int. Cl. G01N 33/28; G01N 21/77

(52) U.S. Cl. CPC G01N 33/2841; G01N 21/7703; G01N 2021/772

(57) ABSTRACT

A hydrogen sensor for detecting hydrogen in a fluid in physical contact with the sensor comprises a sensing element, a first protection layer, provided to prevent contact of the sensing element with a sensor poisoning gas in the fluid, wherein the first protection layer comprises PMMA. Further, a hydrogen detection system, an electrical device having such a system and a method for producing a sensor are provided.

Aspects of the present disclosure relate to a sensor for gas, which has a protective layer to shield the sensor from unwanted components in a fluid surrounding the sensor, in particular to a gas sensor having a protective coating, more particularly to a solid state metal-based hydrogen sensor having such a coating.

### TECHNICAL BACKGROUND

Solid state hydrogen sensors possess typically a catalytic layer which has the function to dissociate the hydrogen molecules into hydrogen atoms. The hydrogen atoms diffuse into the sensing element upon which it changes its physical properties, which can be correlated to the hydrogen concentration of the probed medium. This change can be for example a variation of the resistivity or optical properties of the sensing element and is recorded with an electrical or optical measurement, respectively.

Some gases, for example H₂S, may cause poisoning of this catalytic layer which is reflected in the degradation of the sensing properties. This can lead to a reduction in the lifetime of the sensor and/or to a degradation of measurement accuracy, in particular to wrong detector readings due to a permanent or reversible poisoning of the sensor with such gases. It is therefore important to protect such sensors from contact with poisoning gases. This can be achieved with a protective coating on top of the catalytic layer which allows diffusion of hydrogen but blocks poisoning gases.

A possible application of interest where a protective coating is needed is the detection of hydrogen dissolved in transformer oil. The hydrogen can be taken as an early indicator of transformer faults. It is clear that while an application for measuring dissolved hydrogen in a liquid, particularly in oil is mentioned, the protective layer is also suitable for the use of a sensor for detection in a gas phase.

WO2009/126568 refers to a protective coating for a solid state sensor having a catalytic layer, as e.g. consisting of Pd alloys. The protective layer contains silicon dioxide to protect from contaminating gases, in particular oxygen, which may lead to an oxidation of parts of the sensor.

US2010/0014151 A1 refers to a hydrogen sensor based on a switchable mirror device having a protective coating which is water impermeable. As a protection coating, PTFE is mentioned. The coating has the function to block the transport of liquids, such as water, to the sensor.

EP 2 105 734 A1 discloses a sensor having a gas-sensitive layer with a surface area and an electrical potential sensor capacitively coupled with the area via an air gap, the area being covered by an electric insulating coating that is permeable for hydrogen and oxygen gases. The coating contains polymethylmethacrylate.

WO 2009/126568 A1 discloses a protective coating for sustaining long term performance of a solid-state sensor of a gaseous constituent in a fluid stream. The sensor comprises a catalyst layer for promoting electrochemical dissociation of the gaseous constituent, the coating comprises at least one layer of silicon dioxide.

U.S. Pat. No. 5,783,152 A discloses a sensor probe device for monitoring of hydrogen gas concentrations and temperatures by the same sensor probe. The sensor probe is constructed using thin-film deposition methods for the placement of a multitude of layers of materials sensitive to hydrogen concentrations and temperature on the end of a light transparent lens located within the sensor probe.

U.S. Pat. No. 5,640,470 A discloses a fiber-optic detector for detecting analytes, especially pH estimation in blood. The end of the fiber-optic is coated with a terpolymer like PMMA, which is permeable for hydrogen.

In view of the above and other factors, there is a need for the present invention.

### SUMMARY OF THE INVENTION

In view of the above, a hydrogen sensor according to claim 1, a detection system according to claim 11, a device according to claim 12, and a method according to claim 13 are provided.

Further advantages, features, aspects and details that can be combined with embodiments described herein are evident from the dependent claims, the description and the drawings.

According to a first aspect a hydrogen sensor for detecting hydrogen in a fluid in physical contact with the sensor is provided. The sensor comprises a sensing element, and a first protection layer, provided to prevent contact of the sensing element with a sensor poisoning gas in the fluid, wherein the first protection layer comprises PMMA (polymethyl methacrylate).

According to a further aspect, a detection system for hydrogen in fluids is provided. The detection system comprises a hydrogen sensor for detecting hydrogen in a fluid in physical contact with the sensor, wherein the sensor comprises a sensing element, and a first protection layer, provided to prevent contact of the sensing element with a sensor poisoning gas, wherein the first protection layer comprises PMMA, a temperature sensor, a light source, a light detection device, and a control unit, wherein light from the light source is coupled into the sensor, light reflected by the sensing element of the sensor is detected by the light detection device, and wherein the control unit processes an output signal of the light detection device, determines a hydrogen concentration, and delivers a respective signal.

According to a yet further aspect, a device for electric power generation, transmission, or distribution is provided, comprising an oil volume, and a detection system for hydrogen in the oil, wherein the detection system comprises a hydrogen sensor for detecting hydrogen in a fluid in physical contact with the sensor, wherein the sensor comprises a sensing element, and a first protection layer, provided to prevent contact of the sensing element with a sensor poisoning gas, wherein the first protection layer comprises PMMA, a temperature sensor, a light source, a light detection device, and a control unit, wherein light from the light source is coupled into the sensor, light reflected by the sensing element of the sensor is detected by the light detection device, and wherein the control unit processes an output signal of the light detection device, determines a hydrogen concentration, and delivers a respective signal.

According to a further aspect, a method for producing a hydrogen sensor for detecting hydrogen in a fluid in physical contact with the hydrogen sensor is provided. The method comprises providing a sensing element, and providing a first protection layer comprising PMMA on the sensing element.

It was found that the typical hydrogen concentrations in transformer oil can be more than 10 times smaller than the concentration of carbon monoxide (CO). As CO is a gas which is poisonous for sensors with a Pd-based catalyst, the protection of the hydrogen sensor from CO in a fluid to be probed is of high importance for its technical application.

The advantages of sensors with a protection coating according to embodiments are for example, that the sensor is protected from poisoning induced by carbon monoxide in an effective manner. Further, the sensor is protected from liquids, such as oil, and can hence also be used directly for liquid-dissolved hydrogen detection in applications such as power transformer diagnostics. Also, protection layers comprising PMMA can be combined in multilayer approaches with other coatings such as PTFE (polytetrafluoroethylene), SiO₂ (silicon dioxide), or Aluminium Oxide.

### BRIEF DESCRIPTION OF THE FIGURES

More details will be described in the following with reference to the figures, wherein

FIG. 1 is a schematic view of an examplary hydrogen sensor useful for understanding the invention;

FIG. 2 is a schematic view of a hydrogen sensor according to embodiments;

FIG. 3 is a schematic view of a yet further hydrogen sensor according to embodiments;

FIG. 4 is a diagram of a test with two conventional sensors;

FIG. 5 is a diagram of a test with a sensor according to embodiments;

FIG. 6 is a schematic view of a hydrogen sensor according to embodiments;

FIG. 7 is a schematic view of a hydrogen detection system according to embodiments;

FIG. 8 is a schematic view of an electrical device according to embodiments.

### DETAILED DESCRIPTION OF ASPECTS OF THE INVENTION

As used herein, metal alloys defined by a formula with percent values typically adding to 100 percent, such as, for example, Mg₅₂Ni₂₀Zr₂₈, are meant to include also substances with a composition deviating from that with the exact numbers provided. Typically, alloys having a composition wherein each number, independently from one another, has a tolerance of +/−15 percent, are still regarded to fall under the metal alloy provided by provision of the exact formula, such as the example above, also if the single numbers do not add up to 100 in total. Also, as used herein, such alloys may comprise further, non-named substances such as chemical elements of smaller amounts, such as up to about 2 percent each, but not more than about 10 percent in total.

As used herein, the term “fluid” is intended to be both representative for gases and liquids. It is, however, mainly used to be representative of an insulation liquid, particularly an oil, which is part of the insulation and/or cooling system of an electrical device, more particularly of a power transformer.

In the following, some aspects of the invention are described in detail. Aspects and parts of aspects are independent of each other and can be combined in any manner. For example, any aspect or embodiment described in this document can be combined with any other aspect or embodiment, as long as the combinations achieved are technically feasible.

First, some general possible aspects relating to the sensor assembly are described. The sensor assembly is adapted for sensing a status condition of an insulation-liquid-filled electrical equipment. Herein, electrical equipment refers to any equipment such as shunt reactors, bushings and transformers. The invention is particularly suited for the insulation liquid being insulation oil, be it on a mineral basis or from organic sources, such as palm oil. The invention is further particularly suited for the electrical equipment being a transformer, such as a power or distribution transformer, and more particularly for an oil-filled transformer.

According to aspects, the protective coating of PMMA can be applied to a variety of hydrogen sensor types which have a catalytic layer and may suffer from poisoning issues. This is valid, e.g., for resistive sensors or FET sensors. A typical possible configuration is a fiber-optic hydrogen sensor. Therein, a Pd alloy can serve both as a catalytic layer and a sensing layer.

The PMMA coating is deposited on top of the Pd alloy layer, which is deposited at the end of an optical fiber. The sensor may have e.g. a Ti adhesion layer between the optical fiber and the sensing layer. Other possible fiber optic sensor configurations are related to a multilayer system where the catalytic layer, typically a palladium alloy, is deposited onto a different sensing layer (e.g. MgTi), and the PMMA covers the catalytic layer or both. The optimal thickness of the protective layer can be chosen in a way for having a fast diffusion time of hydrogen through the coating and maximum blocking of CO. Typical coating thicknesses can be e.g. from about 100 nm to about 3.000, more typically from about 150 nm to about 2.000 nm, for a high diffusion rate of hydrogen, but larger thicknesses can be selected if a fast response time for hydrogen detection is not required, and if, for example, a protection is required against significantly higher CO concentrations than are described herein.

Other aspects include the combination of PMMA with other coating materials. For instance, state of the art shows that PTFE (polytetrafluoroethylene) coatings show good properties for the protection on solid state hydrogen sensors towards water. This coating, however, does not protect the sensor from CO. The advantages of both polymeric coatings can be used at the same time by combining them in a multilayer approach. Another suitable coating that can be combined with PMMA is SiO₂. SiO₂ coatings have been shown to prevent oxidation of the catalytic layer in hydrogen sensors. In embodiments, PMMA is thus combined with SiO₂ to prevent both oxygen and CO diffusion. Another possibility is to combine all three coatings together, PMMA, SiO2, and PTFE. The connection between the different layers can be improved by individual activation of the already deposited coating layers by pre-sputtering with Ar ions, for example.

The PMMA coating can be deposited by different techniques on the sensor, e.g. by magnetron sputtering, or by spin-on or chemical vapor deposition techniques. Alternatively, a PMMA socket can be directly slipped on a fiber, or other sensor substrate, and sealed to it. These protective coatings according to aspects are suitable both for detection in the gas phase as well as in liquids, such as, e.g., in transformer oil.

### DETAILED DESCRIPTION OF THE FIGURES AND EMBODIMENTS

It shall be noted that the figures are not drawn to scale, and that some dimensions in the figures are exaggerated for illustrational purposes. The width of the sensor in a horizontal direction is typically much larger, for example 2 to 5 orders of magnitude, than the thickness of the sensing elements and protection layers in a vertical direction (in relation to the drawing plane).

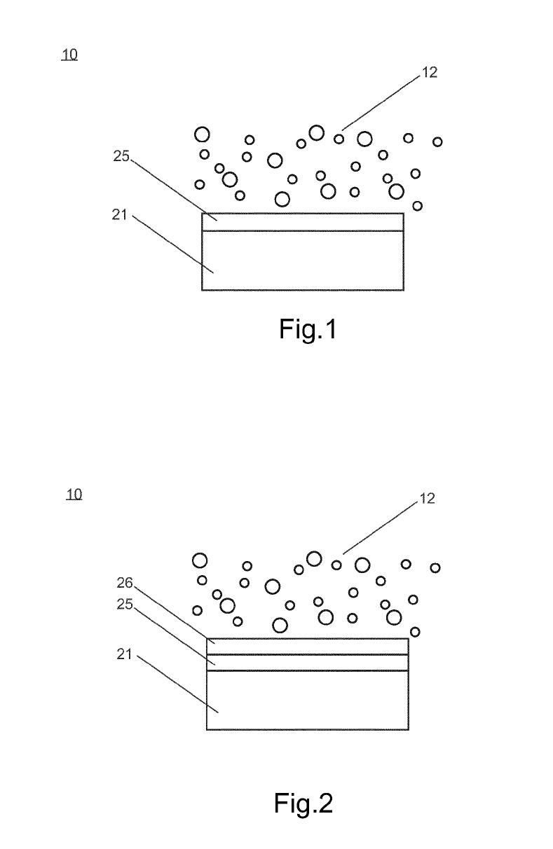

FIG. 1 shows an exemplary hydrogen sensor 10 useful for understanding the invention, for detecting hydrogen in a fluid 12 which is in physical contact with the sensor. A sensing element 21 is covered by a first protection layer 25. The sensing element typically, but not necessarily comprises a catalyst layer and a sensing layer, which can also be identical in the case of the use of Pd alloys. The hydrogen sensor 10 may typically be attached with the sensing layer to an optical fiber (not shown), through which light is directed onto the sensing element 21, while light reflected by the sensing element back into the optical fiber is guided to a light detection device where it is analysed.

The first protection layer 25 prevents direct contact of the sensing element 21 with the fluid 12, but is permeable for hydrogen. The first protection layer 25 comprises PMMA. It has a typical thickness from about 100 nm to about 3.000 nm, more typically from about 150 nm to about 2.000 nm. PMMA has been shown to have good blocking capabilities against CO in the fluid, while at the same time being highly permeable for the hydrogen which needs to pass the protection layer in order to reach the sensing layer.

FIG. 2 shows a hydrogen sensor according to embodiments, wherein a first protection layer 25 of PMMA, as shown in FIG. 1, is accompanied by a second protection layer 26 which abuts the first protection layer 25 and comprises a further, different material. The second protection layer may for example comprise one of PTFE, SiO₂, and Aluminium Oxide, typically Al₂O₃. In the case of PTFE, for example, it is known to have excellent blocking capabilities against liquids, such as water and oil. It is used in embodiments as an outermost coating layer for a hydrogen sensor for use in liquids. In embodiments, it may be employed as the outermost coating layer, wherein a layer comprising PMMA is located between the sensing layer 21 and the PTFE layer.

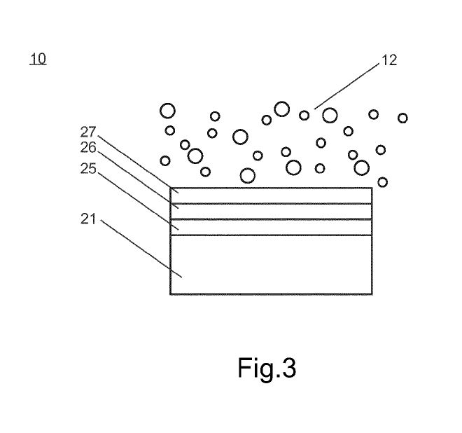

FIG. 3 shows the sensor of FIG. 2 according to embodiments, which comprises a third protection layer 27. The latter abuts the first protection layer 25 or the second protection layer 26. It may comprise one of PTFE, SiO₂, and Aluminium Oxide.

By choosing suitable material combinations for the first protection layer, and optional second protection layer and third protection layer, a hydrogen sensor with high robustness and stability for use in various environments can be designed wherein the environment may also comprise aggressive components. This is particularly interesting for environments with high temperatures, or with a significant amount of aggressive substances in the fluid in contact with the sensor. Thereby, by choosing a suitable combination, the required stability may be achieved while obtaining a high permeability for hydrogen, which is required for efficient operation of the hydrogen sensor 10.

Thereby, a protective layer of SiO₂ is suitable to prevent oxidation of the protected catalytic layer of the sensing element 21 in hydrogen sensors. Thus, when combining a first protection layer of PMMA with a second protection layer of SiO₂, it is possible to prevent both oxygen and CO diffusion from the surrounding fluid 12 to the sensing element 21. Additionally, PTFE may be employed to shield the sensor from liquid water and from oil.

The connection between the sensing element 21 and the first, second and third protection layers can be improved by activation of the already deposited layers by pre-sputtering with Ar ions.

In embodiments, the PMMA coating can be deposited by different techniques on the sensing element. Applicable methods include magnetron sputtering, spin-on deposition or chemical vapor deposition techniques. Also, a PMMA socket can be directly slipped on the fiber and be sealed to it.

The described first, second and third protective layers, respectively coatings, are suitable both for application for detection in the gas phase as well as in liquids. Of particular interest in the present context is the application in insulation liquid of an electrical transformer, also known as transformer oil.

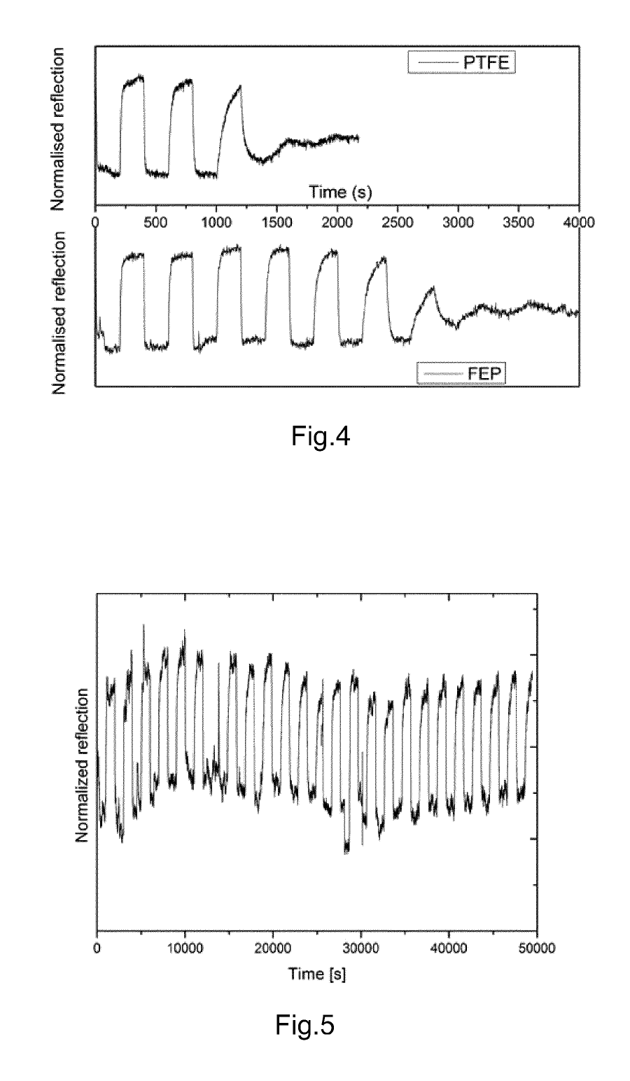

FIG. 4 shows an example of the degradation of the optical response of a hydrogen sensor according to previously known technology, which is a fiber optic sensor with a sensing element wherein the sensing layer and the catalyst layer are one, and comprise a Pd alloy. The sensing layer, more precisely the catalyst layer, is coated with two different polymers for the test, namely PTFE (top in FIG. 4) and Fluorinated Ethylene Propylene (FEP), below in FIG. 4. The optical response changes over time, when the sensor is exposed to a series of hydrogenation cycles, meaning that the hydrogen concentration above the sensor is elevated and reduced to zero alternatingly, resulting in a respective change in reflectance of the fiber optic sensor. The diagrams in FIG. 4 show the intermitting change of reflectance. Due to the presence of CO in the gas in the experiment, the sensor degradates. After several cycles, the optical response of the hydrogen sensor does not change anymore in spite of frequently changing hydrogen concentrations. Thus, in both cases, the PTFE and FEP coatings are not able to protect the sensor from poisoning from CO, even at low CO concentrations, resulting in an almost complete failure of the two conventionally coated sensors after CO exposure for about 1000 seconds in the case of PTFE coating, and after about 2500 seconds in the case of a FEP coating.

As a comparison, FIG. 5 shows a result of the same test as described with respect to FIG. 4, but with the same sensor coated with PMMA according to embodiments, instead of PTFE or FEP. This sensor can be cycled with hydrogen over a long period of time without suffering a degradation of its optical sensing properties by a CO content in the applied gas mixture. This result is even more significant when regarding that the concentration of the potentially sensor poisoning CO content in the gas above the sensor was ten times higher than in the tests for the conventional sensors coated with PTFE and FEP, which were described with respect to FIG. 4. Thus, PMMA as a coating or protection layer against CO poisoning of hydrogen sensors has very good protection properties. At the same time, the diffusion of hydrogen to the sensing layer is not, or only to a negligible extent, influenced by the PMMA protection layer.

In embodiments, the sensing element 21 may be one of a thin film, a FET, a resistive element, and a waveguide. There are a variety of sensing elements known in the art which are suitable to detect hydrogen in a fluid. These can generally be equipped with a protection layer 21 comprising PMMA, and optionally with the second protection layer 26 and third protection layer 27 in the material combinations as described before. In the following, a hydrogen sensor 100 is described, which is a non-limiting example of an optical hydrogen sensor according to embodiments.

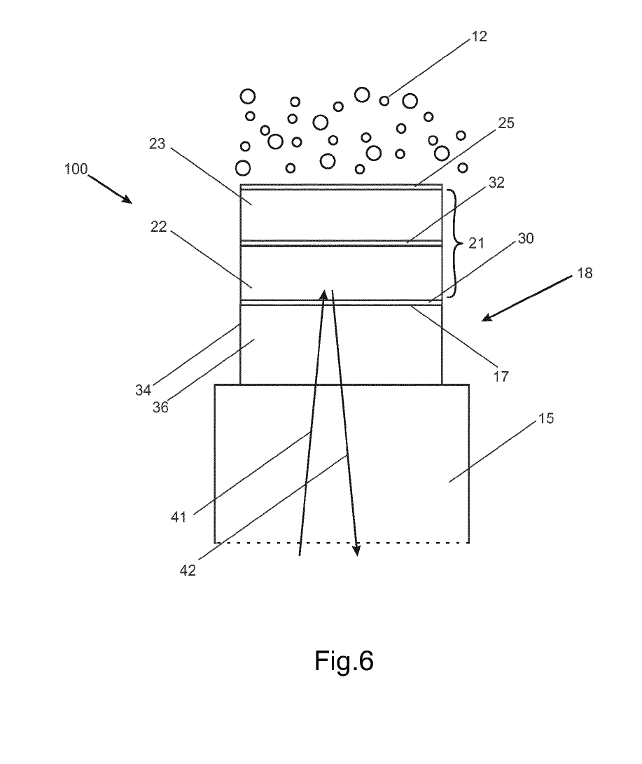

In FIG. 6, an exemplary hydrogen sensor 100 according to embodiments is shown, comprising a sensing element 21 as described above with a sensing layer 22 and a catalyst layer 23. The optical sensor 100 is suitable for detecting hydrogen in a fluid 12 (schematically shown, with smaller circles representing dissolved hydrogen in the fluid, and bigger circles symbolizing CO molecules) which is in physical contact with the hydrogen sensor 100. The sensing element 21, being a multilayer, is coated to an end portion 18 of an optical fiber 15. The latter typically has an outer diameter of about 230 μm including cladding and coating, and 200 μm for the fiber core, but different diameters are also applicable. The sensing layer 22 comprises a thin film of a metal alloy, wherein the metal alloy of the sensing layer 22 may have the following, non-limiting compositions: The alloy comprises Mg, Ni, and a component M, wherein M is at least one of Zr, Ta, and Hf. The alloy has the composition MgₓNi_{y}M_{z}, wherein x is from 40 to 60, y is from 10 to 40, and z is from 10 to 40, in combinations where the numbers add up to 100. The catalyst layer 26 comprises Pd or a Pd alloy.

In embodiments, the sensing element 21 comprises a sensing layer 22 with one of the alloys with the basic composition Mg₅₂Ni₂₀Zr₂₈ and Mg₅₅Ni₂₇Ta₁₈, wherein the individual amount of the components may deviate by +−15%. The hydrogen sensor 100 exhibits a continuous decrease of the optical reflectivity in the visible optical range, when exposed to a growing hydrogen partial pressure in a fluid 12 in contact with the hydrogen sensor 100. The hydrogen sensor 100 of FIG. 6 comprises an auxiliary layer 32 between the catalyst layer 23 and the sensing layer 22, which abuts the sensing layer 22. The auxiliary layer 32 preferably comprises Ti, which is suitable to block atoms from either neighbouring layer from diffusing into the respective other layer. A further auxiliary layer 30, also typically comprising Ti, is provided as an adhesive layer between the core 36 of the optical fiber 15 and the sensing layer 22. In embodiments, the coating layer 25 reaches over the entire multilayer that is also over the circumferential side faces of the multilayer (not shown).

The sensing element 21, which is a multilayer, is typically provided on an end surface 17 of the optical fiber 15, perpendicular to the longitudinal axis of the optical fiber. In embodiments, most or all layers situated consecutively on the end surface 17 overlap over the edge to cover a portion of the circumferential side face 34 of the core 36 of the optical fiber 15. In embodiments, the sensing element 21 may also be provided exclusively on the peripheral side face 34 of the optical fiber 15. Also, the sensing element 21 may in embodiments be provided on an optically transparent substrate (not shown) different from an optical fiber.

A first protection layer 25 comprising PMMA is protecting the sensing element 21 from CO in the fluid 12 in contact with the sensor 100. The first protection layer 25 may be accompanied by a second protection layer 26 and a third protection layer 27 as was described with respect to embodiments shown in FIGS. 2 and 3. Hence, hydrogen sensor 100 may be protected by a multilayer protective layer comprising a combination of layers comprising PMMA, PTFE, SiO₂, and Aluminium Oxide, typically Al₂O₃.

In all embodiments, typical dimensions (i.e., a thickness parallel to the longitudinal axis of the optical fiber) for the varying layers of the multilayer are: Auxiliary layers from 2 to 7 nm, more typically from 4 to 6 nm, for example 5 nm. The sensing layer is typically from 30 to 80 nm, more typically from 40 to 70 nm, for example 60 nm thick. The catalyst layer 26 is typically from 15 nm to 50 nm, more typically from 20 to 40 nm, for example 30 nm thick. The thickness of the coating layer may vary depending on its individual setup, in particular if it comprises several layers of differing materials, as described herein. It may thus have a thickness from about 150 nm to 5 μm, more typically from 20 nm to 3 μm, for example 1 μm or 2 μm.

Schematically, an incoming light beam 41 is shown, which is reflected in the sensing layer 22 and mirrored back into the optical fiber 15 as light beam 42. The optical fiber 15 is typically a multimode fiber, the wavelength may for example be about 635 nm, but also any other wavelength in the visible optical range is applicable with the sensing layers 22 as described herein. The exact position at which the light is reflected in the sensing layer in FIG. 6 is randomly chosen for illustrational purposes only.

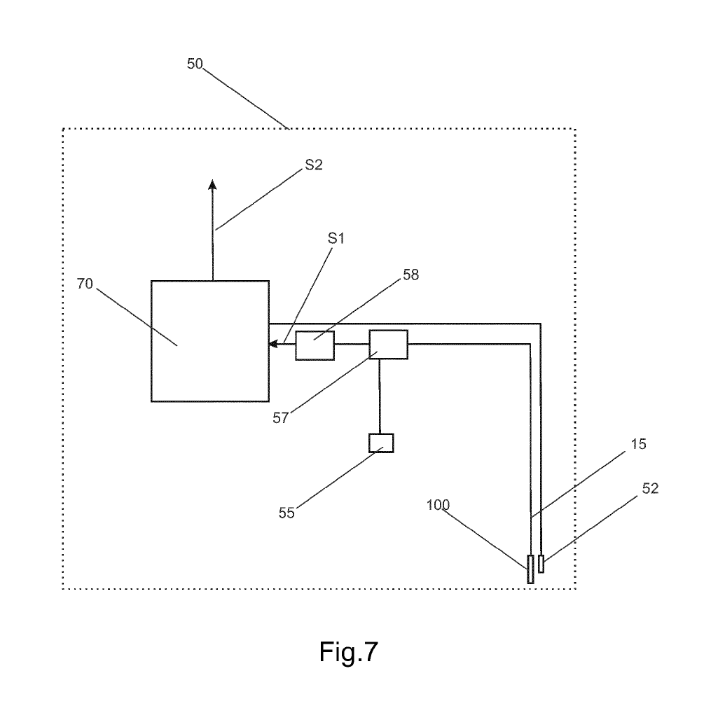

In FIG. 7, a detection system 50 for hydrogen in fluids is shown. It comprises the hydrogen sensor 100 as described above. Further, it comprises a temperature sensor 52, a light source 55, a light detection device 58, an optical splitter 57, and a control unit 70. Light from the light source 55 is coupled by the splitter 57 into the hydrogen sensor 100. In the hydrogen sensor 100, the sensing layer 22 of the sensing element 21 (both not shown) reflects a portion of the incoming light. The reflected light reflects back through the splitter 57 and is detected by light detection device 58. The latter produces an output signal S1 depending on the amplitude of the reflected light received. The control unit 70 processes the output signal S1 of the light detection device 58. It determines a hydrogen concentration and delivers a respective output signal S2. Thereby, it takes into account temperature T delivered from temperature sensor 52.

Thereby, the control unit 70 makes use of data stored in a look-up table. The stored data comprises characteristic reflectance data of the optical sensor 10 for a variety of temperatures (in the range of interest, e.g. 10° C. to 100° C.) and a variety of partial pressures of hydrogen in a fluid 12 (not shown) surrounding the hydrogen sensor 100. Thus, the detection system 50 provides an output signal S2 depending on the calculated hydrogen concentration in the fluid 12, which is calculated from the measured values for temperature and reflectance. Signal S2 is determined from the reflectance of the hydrogen sensor 100 and the temperature. Thereby, the output signal S2 in embodiments is typically a continuous function of the hydrogen concentration in the fluid 12. The detection system typically delivers a continuous change of the output signal S2 in dependency of a hydrogen concentration at the optical sensor, in a temperature region between 5° C. and 150° C. and for hydrogen partial pressures in the fluid 12 between 0.5 ppm and 5.000 ppm, more typically between 2 ppm and 3.000 ppm.

Alternatively to the use of a look-up table, the hydrogen concentration may be calculated by the control unit 70 from a stored function set, taking into account at least the parameters reflectivity of the hydrogen sensor 100 and the temperature from the temperature sensor 52.

It is understood that the described optical sensors 100 and detection systems have to be characterized prior to their use in order to obtain the data mentioned above about the relation between hydrogen partial pressure, temperature and reflectance of the optical sensor.

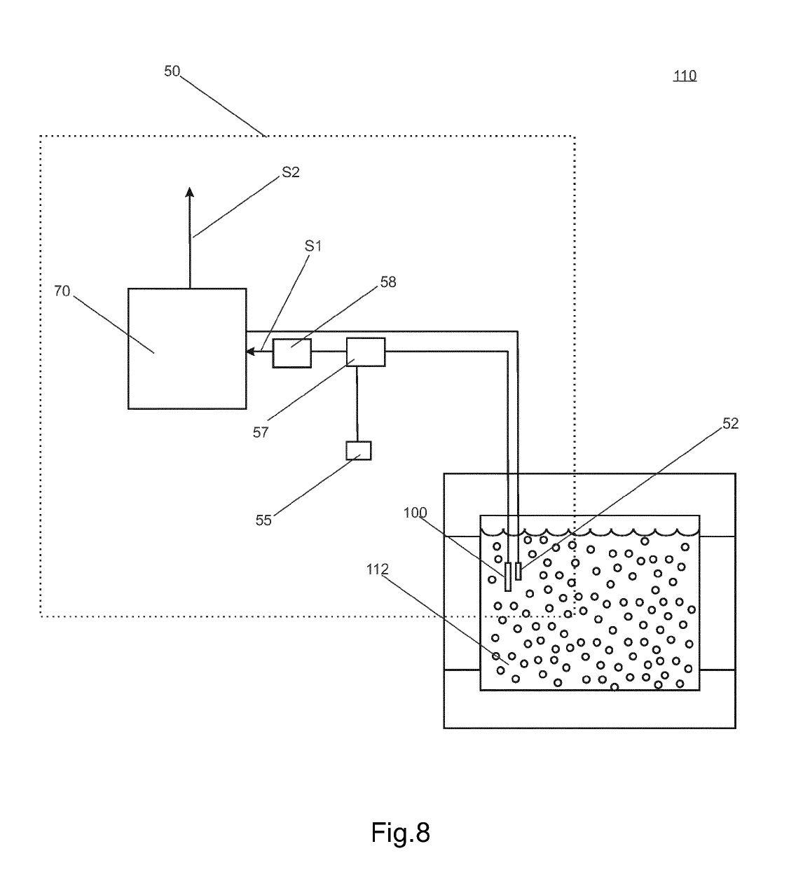

In FIG. 8, an electrical device 110 is shown. It includes a hydrogen detection system 50 as described with respect to FIG. 7. The device 110 is generally a device for electric power generation, transmission, or distribution, and more typically a power transformer or distribution transformer. It comprises an oil volume 112 for insulation and cooling purposes, in which the hydrogen sensor 100 and the temperature sensor 52 are immersed. More precisely, the end portion 18, with the sensing element 21, of the optical fiber 15 is immersed in the oil volume 112. The temperature sensor 52 is provided to measure the temperature of the oil directly at, or adjacent to the sensing element 21 of the hydrogen sensor 100. The oil is representative of the fluid 12 shown in other figures herein.

In embodiments, the hydrogen sensor comprises at least one of a thin film, a FET, a resistive element, and a waveguide as a sensing element. Generally, nearly all types of gas sensors can be protected with one or more protection layers as described. To this end, the sensing element 21 of the sensor is placed in a sputtering device. A layer of PMMA with the intended thickness, as described above, is provided on the sensing layer. During the sputtering process, the PMMA may undergo structural modifications, which may lead to the finding that the deposited PMMA layer has properties different from standard PMMA. Also, it is understood that various other deposition technologies may be employed for providing the PMMA layer, such as spin coating or dipping the fiber end into a solution.

Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. While various specific embodiments have been disclosed in the foregoing, those skilled in the art will recognize that the spirit and scope of the claims allows for equally effective modifications. Especially, mutually non-exclusive features of the embodiments described above may be combined with each other. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A hydrogen sensor for detecting hydrogen in a fluid of oil in physical contact with the sensor, comprising:
an optical fiber with a fiber core,
a multilayer comprising a sensing layer, the multilayer being coated on an end surface of the fiber core perpendicular to a longitudinal axis of the optical fiber,
a first protection layer comprising PMMA, and
a second protection layer comprising one of SiO₂, Aluminium Oxide, and PTFE, wherein the first protection layer and the second protection layer are coated on the multilayer.

2. The sensor of claim 1, wherein the first protection layer has a thickness from 100 nm to 3.000 nm.

3. The sensor of claim 1, comprising a third protection layer abutting the first protection layer or the second protection layer, the third protective layer comprising at least one of PTFE, SiO₂, and Aluminium Oxide.

4. The sensor according to claim 1, wherein the sensing layer comprises a metal alloy.

5. The sensor according to claim 1, wherein the multilayer further comprises a catalyst layer provided between the sensing layer and the first protection layer.

6. The sensor according to claim 1, wherein the multilayer overlaps to cover a portion of the circumferential side face of the fiber core.

7. The sensor according to claim 1, wherein the sensing layer comprises an alloy comprising Mg, Ni, and M, wherein M is at least one of Zr, Ta, and Hf.

8. The sensor of claim 7, wherein the alloy has the composition MgₓNi_{y}M_{z}, and wherein x is from 40 to 60, y is from 10 to 40, and z is from 10 to 40.

9. The sensor according to claim 1, wherein the sensing layer comprises at least one of Mg₅₂Ni₂₀Zr₂₈, Mg₅₂Ni₂₄Zr₂₄, and Mg₅₅Ni₂₇Ta₁₈.

10. The sensor according to claim 1, further comprising at least one auxiliary layer abutting the multilayer.

11. A detection system for hydrogen in fluids of oil, comprising a hydrogen sensor according to claim 1, a temperature sensor, a light source, a light detection device, and a control unit wherein the detection system is adapted such that light from the light source is coupled into the hydrogen sensor, light reflected by the multilayer of the hydrogen sensor is detected by the light detection device, and wherein the control unit is adapted to process an output signal of the light detection device, to determine a hydrogen concentration, and to deliver a respective signal.

12. A device for electric power generation, transmission, or distribution, comprising:
an oil volume; and
a detection system for hydrogen in the oil comprising:
a hydrogen sensor according to claim 1, a temperature sensor, a light source, a light detection device, and a control unit, wherein the detection system is adapted such that light from the light source is coupled into the hydrogen sensor, light reflected by the multilayer of the hydrogen sensor is detected by the light detection device, and wherein the control unit is adapted to process an output signal of the light detection device, to determine a hydrogen concentration, and to deliver a respective signal.

13. A method for producing a hydrogen sensor for detecting hydrogen in a fluid of oil in physical contact with the hydrogen sensor, the method comprising:
providing a multilayer on an end portion of an optical fiber,
coating, on the multilayer, a first protection layer comprising PMMA, and a second protection layer comprising one of SiO₂, Aluminium Oxide, and PTFE.

14. The sensor of claim 2, comprising a third protection layer abutting the first protection layer or the second protection layer, the third protective layer comprising at least one of PTFE, SiO₂, and Aluminium Oxide.

15. The sensor according to claim 5, wherein the catalyst layer comprises Pd.

16. The sensor according to 2, comprising a third protection layer abutting the first protection layer or the second protection layer, the third protective layer comprising at least one of PTFE, SiO₂, and Aluminium Oxide;
wherein the sensing layer comprises a metal alloy;
wherein the multilayer further comprises a catalyst layer provided between the sensing layer and the first protection layer;
wherein the multilayer overlaps to cover a portion of the circumferential side face of the fiber core; and
wherein the sensing layer comprises an alloy comprising Mg, Ni, and M, wherein M is at least one of Zr, Ta, and Hf.

17. The sensor according to claim 16, wherein the sensing layer comprises at least one of Mg₅₂Ni₂₀Zr₂₈, Mg₅₂Ni₂₄Zr₂₄, and Mg₅₅Ni₂₇Ta₁₈; and
further comprising at least one auxiliary layer abutting the multilayer, the auxiliary layer comprising Ti.

18. The sensor of claim 1, comprising a third protection layer abutting the first protection layer or the second protection layer, the third protective layer comprising at least one of PTFE, SiO₂, and Aluminium Oxide;
wherein the sensing layer comprises a metal alloy;
wherein the multilayer further comprises a catalyst layer disposed between the sensing layer and the first protection layer; and
wherein the sensing layer comprises an alloy comprising Mg, Ni, and M, wherein M is at least one of Zr, Ta, and Hf.

19. The sensor according to claim 1, wherein the sensing layer comprises an alloy comprising Mg, Ni, and M, wherein M is at least one of Zr, Ta, and Hf;
wherein the alloy has the composition MgₓNi_{y}M_{z}, and wherein x is from 40 to 60, y is from 10 to 40, and z is from 10 to 40; and
wherein the sensing layer comprises at least one of Mg₅₂Ni₂₀Zr₂₈, Mg₅₂Ni₂₄Zr₂₄, and Mg₅₅Ni₂₇Ta₁₈.

20. The sensor according to claim 10, wherein the auxiliary layer comprising
